(19) United States
(12) Patent Application Publication
(10) Pub. No.: US 2015/0366521 A1
(43) Pub. Date: Dec. 24, 2015

(54) PATIENT SUPPORT SYSTEM

(71) Applicant: Elekta Limited, West Sussex (GB)

(72) Inventor: Mihai TRUTA, WEST SUSSEX (GB)

(73) Assignee: Elekta Limited

(21) Appl. No.: 14/744,989

(22) Filed: Jun. 19, 2015

(30) Foreign Application Priority Data
Jun. 20, 2014 (GB) 1411011.8

(51) Int. Cl. A61B 6/04; A61G 13/04

(52) U.S. Cl. CPC A61B 6/0407; A61G 13/04

(57) ABSTRACT

A patient support system, comprises a patient support having a base and an upper surface, the upper surface moveable relative to the base in a direction along at least one of a first axis, a second axis orthogonal to the first axis, and a third axis orthogonal to the first and second axes; and a control unit for controlling movement of the upper surface, the control unit defining a longitudinal axis. The direction of movement of the upper surface relative to the base is determined by the orientation of the longitudinal axis of the control unit relative to the patient support.

### CROSS-REFERENCE TO RELATED APPLICATION

The present application claims the benefit of priority to United Kingdom Patent Application No. 1411011.8, filed Jun. 20, 2014, the entire content of which is incorporated herein by reference.

### FIELD OF THE INVENTION

This invention relates to a patient support system and, in particular, to a means for manipulating the patient support system.

### BACKGROUND TO THE INVENTION

A patient support may be used in a medical environment to support a patient who is undergoing examination or a medical procedure, for example a radiotherapy procedure.

A known patient support includes an upper surface upon which a patient can sit or lie while a procedure is carried out. The upper surface may be mounted upon a base and may include functionality to enable the upper surface of the support to be moved relative to the base of the support. For example, a patient may need to be positioned so that a body portion, for example the left lung, is positioned over the centre of the base of the patient support. This may be achieved by translating the upper surface of the support to the right with respect to the base, so that the left lung of the patient is positioned centrally relative to the base of the support.

Existing control systems used for manipulating the position of a bed of a patient support or manipulating the position of the entire patient support can be complicated and the use of such control systems is often not intuitive. Thus, it can be difficult to move the patient into the required position.

### SUMMARY OF INVENTION

A first aspect of the invention provides a patient support system, comprising a patient support having a base and an upper surface, the upper surface moveable relative to the base in a direction along at least one of a first axis, a second axis orthogonal to the first axis, and a third axis orthogonal to the first and second axes; and a control unit for controlling movement of the upper surface, the control unit defining a longitudinal axis; wherein the direction of movement of the upper surface relative to the base is determined by the orientation of the longitudinal axis of the control unit relative to the patient support.

When the control unit is oriented such that its longitudinal axis is parallel to or within a defined angular range of the first axis, the upper surface may be moveable along the first axis. When the control unit is oriented such that its longitudinal axis is parallel to or within a defined angular range of the second axis, the upper surface may be moveable along the second axis. When the control unit is oriented such that its longitudinal axis is parallel to or within a defined angular range of the third axis, the upper surface may be moveable along the third axis.

When the control unit is oriented such that its longitudinal axis is outside the defined angular ranges, control of the movement of the upper surface by the control unit may be prohibited. Alternatively, when the control unit is oriented such that its longitudinal axis is outside the defined angular ranges, the upper surface may be moveable in a direction corresponding to a combination of movements along two or more of the first, second and third axes. For example, when the control unit is oriented such that its longitudinal axis has components in two or more of the first, second and third axes (and outside the defined angular ranges of those axes), the upper surface may be moveable in a direction having respective components along the two or more of the first, second and third axes.

The defined angular range about each axis may be a range of 30 degrees.

The control unit may comprise a first user interface for receiving a user input. The upper surface may be configured to move in response to a user input received via the first user interface.

The first user interface may comprise a sliding input device, the sliding input device moveable along a track.

The control unit may have a front end and a rear end. The sliding input device may be movable between a front end of the track and a rear end of the track, the front end and the rear end of the track corresponding to the front end and the rear end of the control unit respectively.

The sliding input device may be biased towards a central position along the track.

When the control unit is oriented such that its longitudinal axis is parallel to or within the defined angular range of a particular one of the first, second and third axes, the direction of movement of the upper surface along the particular axis may correspond to a direction of movement of the sliding input device along the track.

The upper surface may be rotationally moveable relative to the base about at least one of the first axis, the second axis, and the third axis.

The control unit may further comprise a toggle switch, moveable between a first position and a second position. When the toggle switch is in the first position, the first user interface may be configured to effect linear movement of the upper surface and, when the toggle switch is in the second position, the first user interface may be configured to effect rotational movement of the upper surface.

When the control unit is oriented such that its longitudinal axis is parallel to or within the defined angular range of the first axis, the upper surface may be rotatable about one of first, second or third axes. When the control unit is oriented such that its longitudinal axis is parallel to or within the defined angular range of the second axis, the upper surface may be rotatable about another of the first, second or third axes. When the control unit is oriented such that its longitudinal axis is parallel to or within the defined angular range of the third axis, the upper surface may be rotatable about the other of the first, second or third axes.

The sliding input device may be rotatable with respect to the control unit. The sliding input device may be configured such that rotating the sliding input device effects rotation of the upper surface relative to the base of the patient support.

The control unit may include at least one transmitter and the patient support may include at least one receiver. The at least one transmitter may be configured to communicate with the at least one receiver in order for the orientation of the control unit relative to the patient support to be determined

The at least one transmitter may comprise an ultrasound transmitter, infrared transmitter and/or an RFID transmitter and the at least one receiver may comprise an ultrasound receiver, an infrared receiver and/or an RFID receiver. The control unit may comprise an inertial measurement device for determining one or more of the orientation and position of the control unit relative to the patient support. The inertial measurement device may comprise one or more of an accelerometer, a gyroscope, a compass and a magnetometer. The position and orientation of the control unit may be determined by one or more external sensors. Similarly, the position and orientation of the patient support may be determined by one or more external sensors.

The control unit may comprise an activation switch moveable between a first position and a second position, and biased towards the second position. When the activation switch is in the first position, functionality of the first user interface may be enabled and, when the activation switch is in the second position, functionality of the first user interface may be prevented.

It will be appreciated that features of the various aspects of the invention may be combined with those of other aspects of the invention.

### BRIEF DESCRIPTION OF THE DRAWINGS

Embodiments of the invention will now be described, strictly by way of example only, with reference to the accompanying drawings, of which:

FIG. 1A is a perspective view of a patient support system;

FIG. 1B is a diagram showing possible directions of movement of the patient support system of FIG. 1A;

FIGS. 2A and 2B are perspective views of a control unit for manipulating the position of a patient support system;

FIG. 3 is a schematic view of a patient support system and a control unit constructed in accordance with an embodiment of the invention;

FIG. 4 is a schematic view of a patient support system and a control unit constructed in accordance with an alternative embodiment of the invention;

FIGS. 5, 6, and 7 are schematic views of a patient support system and a control unit constructed in accordance with various embodiments of the invention;

FIG. 8A is a side view of a control unit constructed in accordance with an embodiment of the invention; and

FIG. 8B is a perspective view of the control unit of FIG. 8A.

### DESCRIPTION OF THE EMBODIMENTS

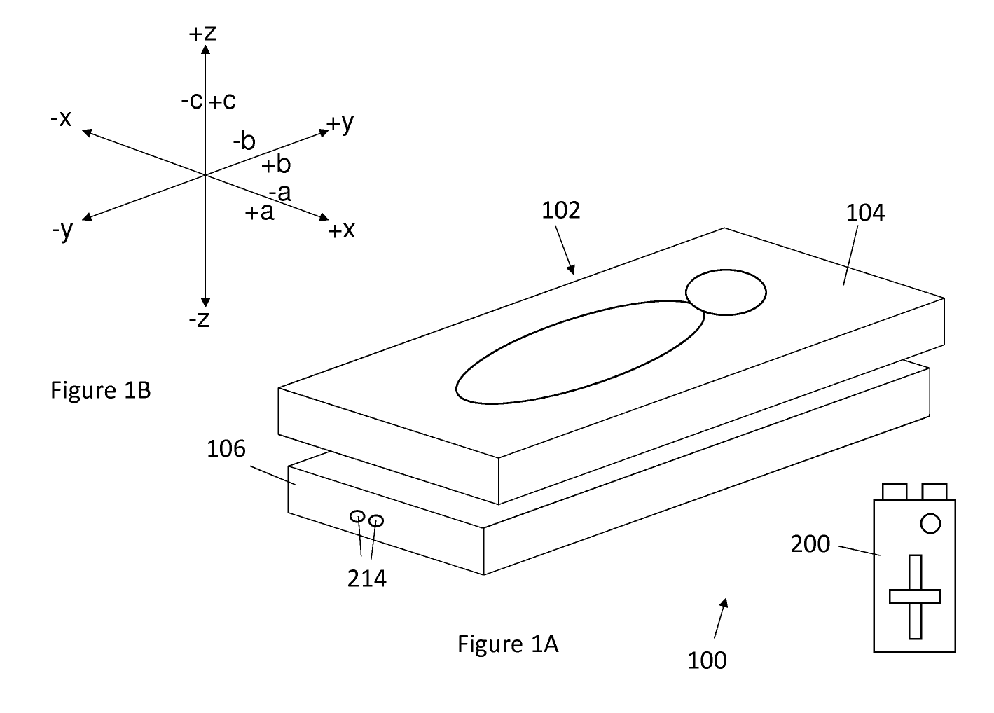

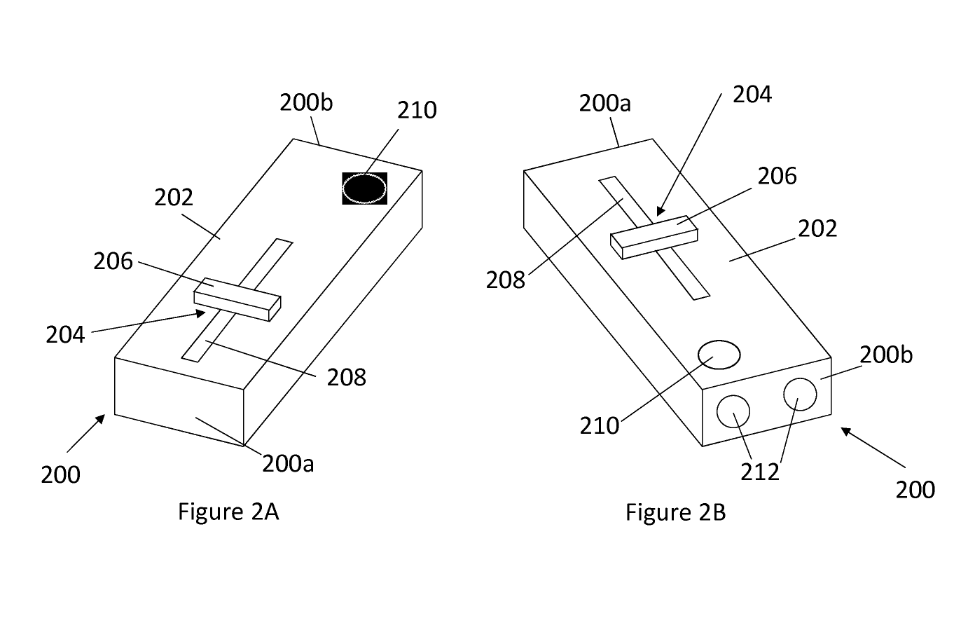

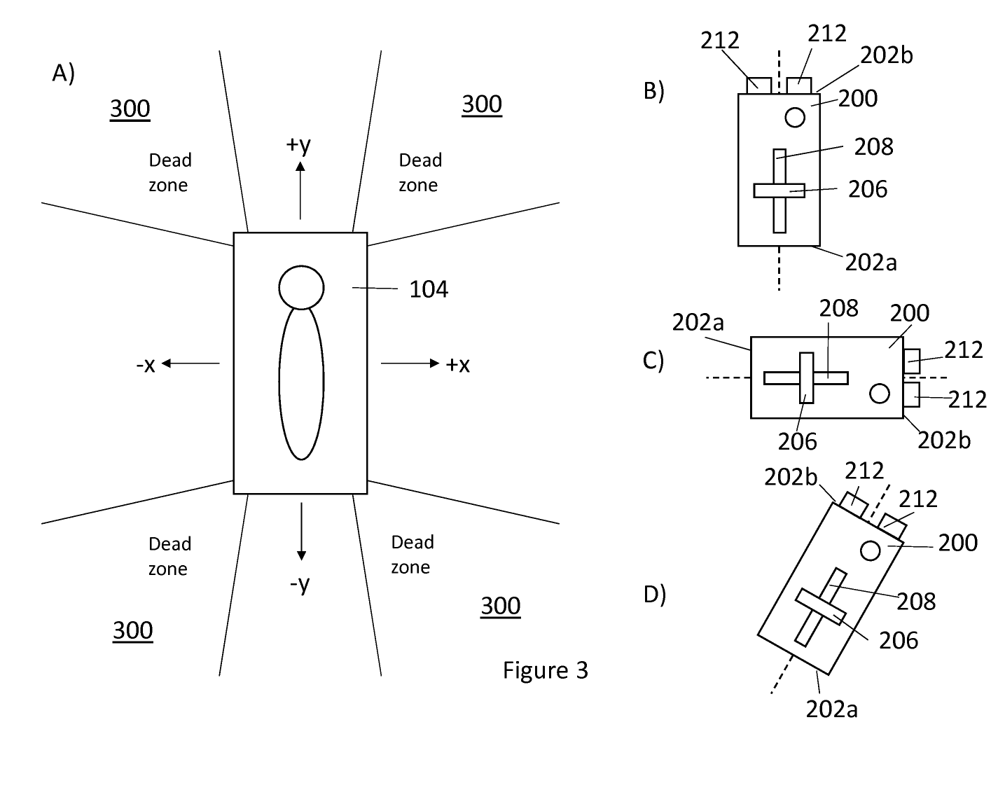

Referring to the drawings, FIG. 1 shows a patient support system 100 that includes a patient support 102 having an upper surface 104 on which a patient can sit or lie down for examination or for treatment. The upper surface 104 is moveably mounted onto a base 106. The upper surface 104 may be moved relative to the base 106 by a mechanism (not shown) located between, or within one or more of, the upper surface and the base. The base 106 may also be moveable, for example along a surface, or adjustable, for example to increase or reduce the height of the upper surface 104 above the ground. Movement of the upper surface 104 relative to the base 106 is achieved using a control unit 200 which may be connected to the patient support 102 by a cable or wirelessly using a known wireless communication standard such as IEEE 802.11a, 802.11g, 802.11n, Bluetooth or RFID. The control unit 200 will be discussed in more detail with reference to FIGS. 2A and 2B.

Depending on the use of the patient support system, it may be necessary to accurately move the patient being examined or treated in one or more number of directions. FIG. 1B shows the possible directions of movement of the upper surface 104 relative to the base 106 and the possible rotational movements of the upper surface. Movement in the +z direction represents a linear movement of the upper surface 104 upwards, away from the base 106, and movement in the −z direction represents a linear movement of the upper surface downwards, towards the base. Movement in the +y direction represents a linear movement of the upper surface 104 in that direction relative to the base 106, and movement in the −y direction represents a linear movement of the upper surface in that direction relative to the base. In this embodiment, and using this notation, it will be appreciated that movement in the +y and −y directions represent linear movement along a longitudinal axis of the patient support 102, and perpendicular to the x and z axes. Movement in the +x direction represents a linear movement of the upper surface 104 in that direction relative to the base 106, and movement in the −x direction represents a linear movement of the upper surface 104 in that direction relative to the base 106. Again, in this embodiment, and using this notation, it will be appreciated that movement in the +x and −x directions represents movement of the upper surface 104 in a direction perpendicular to the y and z axes.

The base 106 may include a mechanism (not shown) to enable it to extend or retract in the +z or −z directions, along the z axis, for example to increase its height. Similarly, movement of the upper surface 104 along the z axis may be achieved by extending or retracting the base along the z axis, while the upper surface 104 is not moved relative to the base.

In addition to linear movement of the upper surface 104 relative to the base 106, rotation of the upper surface relative to the base can also be effected. A rotation in the +a direction represents a rotational movement of the upper surface 104 about the x axis, so as to raise the head of a patient lying on the bed and to lower the feet of the patient. A movement in the −a direction represents a rotational movement of the upper surface 104 relative to the base 106 about the x axis so as to raise the feet of a patient lying on the upper surface and to lower the patient's head. Movement in the +b direction represents a rotational movement of the upper surface 104 relative to the base 106 about the y axis, and movement in the −b direction represents a rotational movement of the upper surface in a direction opposite to the +b direction. Movement in the +c direction represents a rotational movement of the upper surface 104 relative to the base 106 about the z axis, and movement in the −c direction represents a rotational movement of the upper surface about the z axis in a direction opposite to the +c direction.

Rotation in the +a and −a directions about the x axis can be considered to adjust the pitch of the upper surface 104; rotation in the +b and −b direction about the y axis can be considered to adjust the roll of the upper surface; and rotation in the +c and −c directions about the z axis can be considered to adjust to yaw of the upper surface.

FIGS. 2A and 2B show the control unit 200 from two different angles; FIG. 2A shows a rear end 200a of the control unit in a perspective view, and FIG. 2B shows a front end 200b of the control unit in a perspective view. In the described embodiment, the control unit 200 is a handheld unit intended to be held in one hand of an operator, such as technician or medical professional. The control unit 200 has a housing 202 which, in the embodiment shown, is generally rectangular cuboidal in shape. In other embodiments, however, the housing 202 may be formed in a more ergonomic shape, for example to fit comfortably into the hand of an operator.

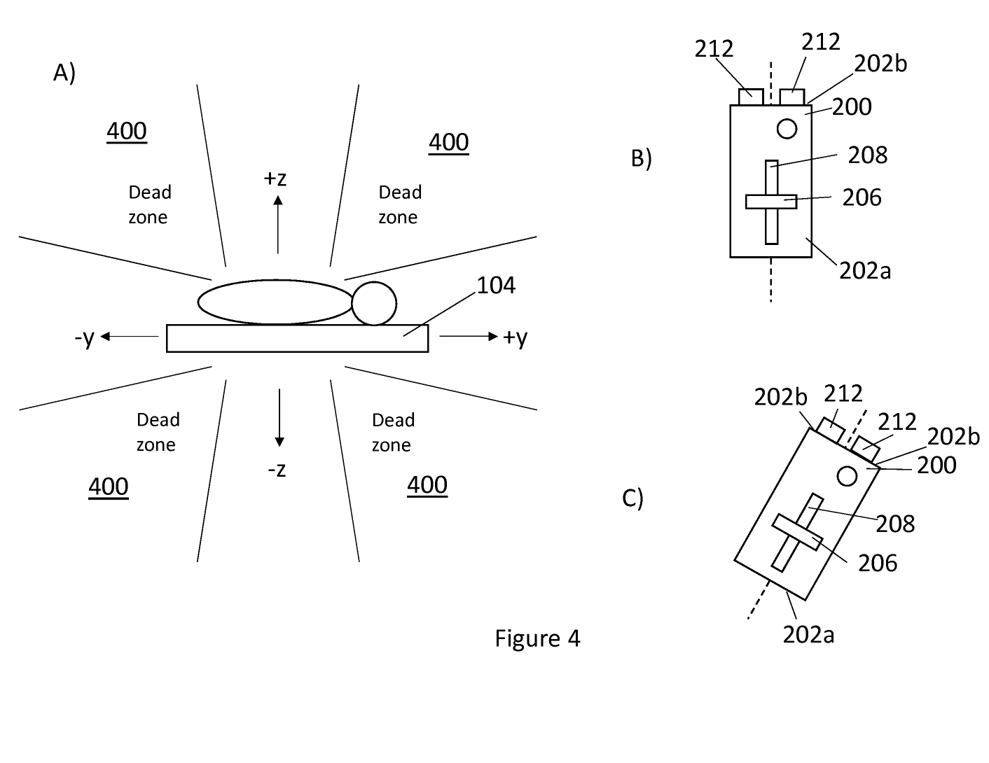

The control unit 200 has two interfaces via which a user can input commands. A first interface 204 is, in this embodiment, in the form of a slider switch which includes an actuator 206 moveable along a track 208. In one embodiment, biasing means (not shown) within the housing 202 act to urge the actuator 206 to a central position along the track 208 such that, when an operator removes his or her finger from the actuator, the actuator returns to its central position along the track, as shown in FIGS. 2A and 2B. An operator is able to slide the actuator 206 in either direction (that is, towards the rear end 200a or towards the front end 200b) along the track 208 in order to effect movement of the upper surface 104 of the patient support 102, as will be discussed with reference to FIGS. 3 to 7.

A second user interface 210, in this embodiment, takes the form of a button which functions as a toggle switch. The button 210 enables an operator to toggle between linear movement and rotational movement. That is to say, when the button 210 is pressed, the first user interface is configured to effect rotational movement of the upper surface 104 (in directions +a, −a, +b, −b, +c and −c), and when the button is not pressed, the first user interface can be used to effect linear movement of the upper surface (in directions +x, −x, +y, −y, +z and −z). Use of the control unit 200 to effect movement of the upper surface 104 will be discussed in greater detail with reference to FIGS. 3 to 7.

The control unit 200 may include an indicator, such as a light (not shown), to indicate to an operator when the button 210 is depressed. For example, the light may be illuminated when the button 210 is depressed and, therefore, causes the first user interface to effect rotational movement of the patient support 102.

With reference to FIG. 2B, a pair of ultrasound emitters 212 are provided on the front end 200b of the control unit 200. In other embodiments, the ultrasound emitters 212 may be located elsewhere on the control unit 200. The ultrasound emitters 212 are configured to communicate with a pair of ultrasound receivers 214 (see FIG. 1A) located on the base 106 of the patient 102. In some embodiments, the base 16 includes multiple pairs of ultrasound receivers 214 which are in communication with the ultrasound emitters 212. In this embodiment, a single pair of ultrasound receivers 214 is shown located on an end of the base 106. However, it will be appreciated that the ultrasound receivers 214 may, in other embodiments, be located elsewhere on the patient support 102, for example on another surface of the base or on the upper surface 104. The purpose of the ultrasound emitters 212 and the ultrasound receivers 214 is to enable a determination to be made of the approximate location of the control unit 200 relative to the patient support 102 and of the orientation of the control unit relative to the patient support.

The control unit 200 may additionally or alternatively include radio frequency identification (RFID) beacons or emitters, which can communicate with RFID trackers or receivers on the patient support 102, the base 106, or elsewhere near to the patient support, in order to determine the orientation of the control unit with respect to the base 106.

In an alternative embodiment, the control unit may include infrared emitters which can communicate with infrared receivers on the patient support 102, the base 106, or elsewhere near to the patient support, in order to determine the orientation of the control unit with respect to the base 106.

In addition to the ultrasound emitters 212 and the ultrasound receivers 214, the control unit 200 may, in some embodiments, include one or more additional sensors such as an inertial measurement device (not shown) to enable a determination to be made of the orientation of the control unit 200 relative to the patient support 102. The inertial measurement device may comprise one or more of an accelerometer, a gyroscope, a compass and a magnetometer.

It will be appreciated that, in alternative embodiments, fewer or more emitters and transmitters may be used. Thus, in the illustrated embodiment, at least one emitter/transmitter is configured to communicate with at least one receiver.

In alternative embodiments, an external sensor system (not illustrated) may be used to track one or more of the orientation and position of the control unit 200, and one or more of the orientation and position of the patient support 102. The external sensor system may then compare the position and/or orientation of the control unit 200 relative to the position and/or orientation of the patient support 102, and thus determine the direction of movement of the upper surface 104 as controlled by the control unit 200 (for which, see below).

It will be appreciated that communication between the control unit and the patient support may be achieved using a wired or cable connection (not shown).

It will be appreciated that, while emitters 212 and receivers 214 communicate with each other using ultrasound, in other embodiments, different communication modalities may be used.

Movement of the upper surface 104 of the patient support 102 using the control unit 200 will now be discussed with reference to FIGS. 3, 4, 5, 6 and 7.

FIGS. 3A to 3D show, diagrammatically, how the upper surface 104 of the patent support 102 may be moved along the x and y axes. FIG. 3A shows the upper surface 104 as viewed from above. The direction of linear movement of the upper surface 104 is determined by the orientation of the control unit 200 relative to the patient support 102. The control unit 200 is considered to define a longitudinal axis and, in order for the control unit to effect linear movement of the upper surface 104 one of the x or y axes, the control unit must be oriented such that its longitudinal axis is parallel to or substantially parallel to that axis along which movement is desired. For example, to enable the control unit 200 to be used to move the upper surface 104 along the y axis, the control unit must be held in such an orientation that its longitudinal axis is parallel to or substantially parallel to the y axis, as shown in FIG. 3B. The control unit is considered to be oriented substantially parallel to a particular axis if it is oriented within a defined angular range of that axis, for example within a 30 degree range. The orientation of the control unit 200 relative to the upper surface 104 is determined by the communication between the ultrasound emitters 212 on the control unit and the ultrasound receivers 214 on the base 106 of the patient support 102. For example, the time-of-flight of signals emitted from each of the emitters 212 and detected by each of the receivers 214 can be used to determine the distance of each emitter from each receiver. This information, combined with the known distances between the emitters 212 and between the receivers 214, allows the relative orientation of the control unit 200 with respect to the patient support 102 to be determined

By including the emitters and receivers in pairs, it is possible to differentiate between the signals transmitted and received by each transmitter/receiver in a pair. The transmitters in a pair are positioned sufficiently far apart from one another that a determination can be made of how the transmitters are positioned relative to one another and, therefore, how the control unit 200 is oriented with respect to the patient support 102. Alternatively, the transmitters in a pair can communicate using different frequencies in order to be distinguished from each other, and thus determine how the control unit 200 is oriented with respect to the patient support 102. In an alternative embodiment, as is mentioned above, the orientation of the control unit 200 may additionally or alternatively be determined using one or more sensors, such as an inertial measurement device located within the housing 202 of the control unit, or RFID beacons located in the control unit and RFID trackers located on or near to the base.

With the control unit 200 oriented in the manner shown in FIG. 3B (that is parallel to or substantially parallel to the y axis), a user is able to move the upper surface in the +y direction by sliding the actuator 206 along the track 208 in the +y direction (that is towards the front end 202b of the control unit), and is able to move the upper surface in the −y direction by sliding the actuator along the track in the −y direction (that is towards the rear end 202a of the control unit).

In this embodiment, the control unit 200 is oriented parallel to the y axis, with the front end 202b of the control unit aimed in the +y direction. If the control unit is held facing the opposite direction, with the front end 202b of the control unit aimed in the −y direction, then the direction of movement effected by the actuator 206 is reversed; sliding the actuator along the track 208 towards the front end of the control unit will cause the upper surface 104 to move in the −y direction. Similarly, movement of the actuator 206 along the track 208 towards the rear end 202a of the control unit 200 will cause the upper surface 104 to move in the +y direction.

In order to move the upper surface 104 along the x axis, the control unit 200 must be oriented parallel to or substantially parallel to the x axis, as shown in FIG. 3C. When the control unit 200 is oriented in this way, a user is able to move the upper surface 104 of the patient support 102 in the +x direction by sliding the actuator 206 along the track 208 in the +x direction (that is towards the front end 202b of the control unit) and is able to move the upper surface in the −x direction by sliding the actuator along the track in the −x direction (that is towards the rear end 202a of the control unit).

In this embodiment, the control unit 200 is oriented parallel to the x axis, with the front end 202b of the control unit aimed in the +x direction. If the control unit is held facing the opposite direction, with the front end 202b of the control unit aimed in the −x direction, then the direction of movement effected by the actuator 206 is reversed; sliding the actuator along the track 208 towards the front end of the control unit will cause the upper surface 104 to move in the −x direction. Similarly, movement of the actuator 206 along the track 208 towards the rear end 202a of the control unit 200 will cause the upper surface 104 to move in the +x direction.

In general, therefore, if the control unit 200 is oriented parallel to or substantially parallel to an axis, then movement of the actuator 206 along the track 208 in a particular direction will cause the upper surface 104 to move in a corresponding direction along the axis.

To ensure that the upper surface 104 will only move in a particular direction when the control unit 200 is oriented substantially parallel to that direction, the patient support system 100 is configured, in one embodiment, such that the upper surface will not move if the control unit is not oriented parallel to or substantially parallel to the direction of movement. Thus, if the controller 200 is oriented such that it is not parallel to or within a defined angular range of a direction of motion (that is, if the control unit is not oriented parallel to or within a defined angular range of the x or y axes), such as in FIG. 3D, then sliding the actuator 206 along the track 208 will not cause any movement of the upper surface 104. The defined angular range may be +/−30 degrees (that is, an arc of 30 degrees either side of the axis). Preferably, the angular range will be no less than +/−10 degrees. Regions 300 in FIG. 3A denote so called “dead zones”. If the control unit 200 is oriented such that its longitudinal axis is parallel to an axis within a dead zone 300, then movement of the actuator 206 along the track 208 will have no effect on the position of the upper surface 104.

Alternatively, when the control unit 200 is oriented such that its longitudinal axis is outside the defined angular ranges, the upper surface may be moveable in a direction corresponding to a combination of movements along two or more of the first, second and third axes. For example, when the control unit is oriented such that its longitudinal axis has components in two or more of the first, second and third axes (and outside the defined angular ranges of those axes), the upper surface may be moveable in a direction having respective components along the two or more of the first, second and third axes.

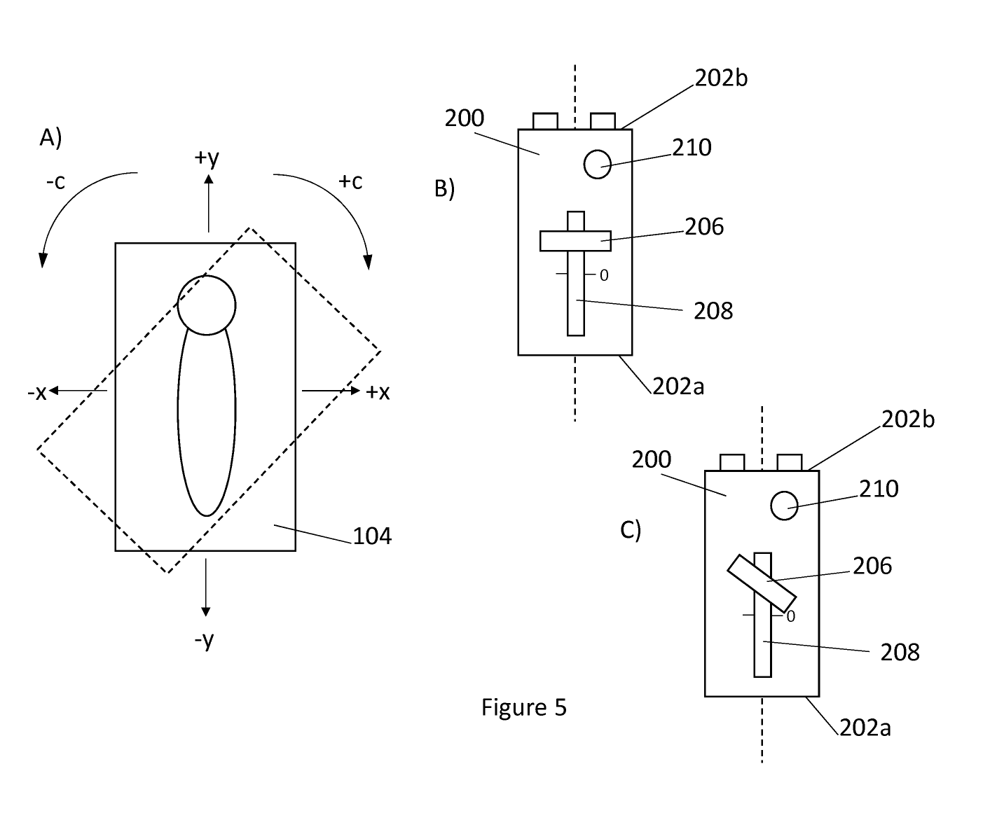

FIGS. 4A to D show diagrammatically how the upper surface 104 of the patent support 102 may be moved along the y and z axes. FIG. 4A shows the upper surface 104 as viewed from the side. As is discussed above, in order for the control unit 200 to effect linear movement of the upper surface 104 one of the y or z axes, the control unit must be oriented such that its longitudinal axis is parallel to or substantially parallel to that axis along which movement is desired. In other words, to enable the control unit 200 to be used to move the upper surface 104 along the z axis, the control unit must be held in an orientation parallel to or substantially parallel to the z axis, as shown in FIG. 4B.

With the control unit 200 oriented in the manner shown in FIG. 4B (substantially parallel to the z axis), a user is able to move the upper surface in the +z direction by sliding the actuator 206 along the track 208 in the +z direction (that is towards the front end 202b of the control unit), and is able to move the upper surface in the −z direction by sliding the actuator along the track in the −z direction (that is towards the rear end 202a of the control unit).

In this embodiment, the control unit 200 is oriented parallel to the z axis, with the front end 202b of the control unit aimed in the +z direction. If the control unit is held facing the opposite direction, with the front end 202b of the control unit aimed in the −z direction, then the direction of movement effected by the actuator 206 is effectively reversed; sliding the actuator along the track 208 towards the front end of the control unit will cause the upper surface 104 to move in the −z direction. Similarly, movement of the actuator 206 along the track 208 towards the rear end 202a of the control unit 200 will cause the upper surface 104 to move in the +z direction.

Again, if the controller 200 is oriented such that it is not parallel to a direction of motion (that is, if the control unit is not oriented parallel to the y or z axis), such as in FIG. 4C, then sliding the actuator 206 along the track 208 will not cause any movement of the upper surface 104. Regions 400 in FIG. 4A denote so called “dead zones”. If the control unit 200 is oriented such that its longitudinal axis is parallel to an axis lying within a dead zone 400, then movement of the actuator 206 along the track 208 will have no effect on the position of the upper surface 104.

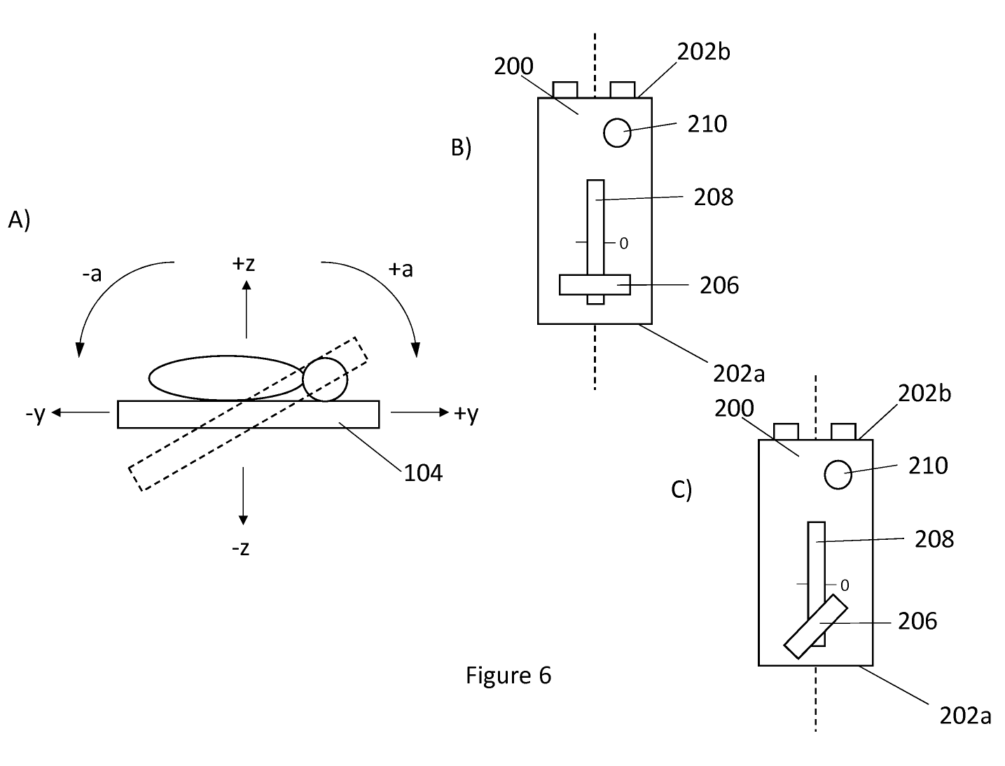

FIGS. 5A and 5B show how the upper surface 104 of the patient support 102 may be rotated in the +c and −c directions, about the z axis. In FIG. 5A, the upper surface 104, shown in plan view, is oriented with its longitudinal axis parallel to the y axis. As is mentioned above, to effect rotational movement of the upper surface 104, the button 210 on the control unit 200 must be pressed which, effectively, puts the control unit into rotation mode. With rotation mode selected, the actuator 206 can be used to rotate the upper surface 104 in the +c and −c directions. In this embodiment, with the control unit 200 oriented as shown in FIG. 5B, along the y axis, and with the front end 202b aimed in the +y direction, then sliding the actuator in the +y direction (towards the front end of the control unit, as shown in FIG. 5B) causes the upper surface to rotate in the +c direction towards the position shown by a dashed line. Conversely, sliding the actuator 206 along the track 208 in the −y direction (towards the rear end 202a of the control unit 200) causes the upper surface 104 to rotate in the −c direction.

In this embodiment, orienting the control unit 200 in the opposite way, by aiming the front end 202b in the −y direction, does not change the functionality of the actuator 206; with the control unit 200 oriented in this way (that is with the front end 202b aimed in the −y direction), sliding the actuator in the −y direction (towards the front end of the control unit) will cause the upper surface 104 to rotate in the +c direction, and sliding the actuator in the +y direction (towards the rear end 202a of the control unit) will cause the upper surface to rotate in the −c direction. In other words, when the control unit is in rotation mode, and is oriented substantially parallel to the y axis, sliding the actuator 206 towards the front end 202b of the control unit causes the upper surface 104 to rotate clockwise when viewed from above (as in FIG. 5A).

In an alternative embodiment, as shown in FIG. 5C, the actuator 206 is rotatable with respect to the control unit 200 and/or to the track 208. For example, the actuator may be rotatable around its point of contact with the track 208. In some embodiments, this rotation may only be possible once the button 210 is depressed. In other embodiments, rotation may be possible when the button 210 is not depressed, but no rotation signal is transmitted unless the button is depressed. If the control unit is oriented substantially parallel with respect to the y axis, then rotating the actuator 206 in the +c direction will cause the upper surface 104 of the upper surface 104 of the patient support to rotate in the +c direction. Similarly, rotating the actuator in the −c direction will cause the upper surface 104 of the patient support to rotate in the −c direction. Releasing the button 210 will allow the user to return the actuator 206 to a neutral position with respect to the track 208 without moving the upper surface 104. This may be useful when further adjustments to the position of the upper surface are necessary after the upper support has been rotated.

FIGS. 6A and 6B show the upper surface 104 of the patient support 102 may be rotated in the +a and −a directions, about the x axis. In FIG. 6A, the upper surface 104, shown in a side view, is oriented with its longitudinal axis parallel to the y axis. With rotation mode selected, the actuator 206 can be used to rotate the upper surface 104 in the +a and −a directions. In this embodiment, with the control unit 200 oriented as shown in FIG. 6B, along the z axis, and with the front end 202b aimed in the +z direction, then sliding the actuator in the +z direction (towards the front end of the control unit) causes the upper surface to rotate in the +a direction. Conversely, sliding the actuator 206 along the track 208 in the −z direction (towards the rear end 202a of the control unit 200, as shown in FIG. 6B) causes the upper surface 104 to rotate in the −a direction, towards the position shown by the dashed line in FIG. 6A.

In this embodiment, orienting the control unit 200 in the opposite way, by aiming the front end 202b in the −z direction, does not change the functionality of the actuator 206; with the control unit 200 oriented in this way (that is with the front end 202b aimed in the −z direction), sliding the actuator in the −z direction (towards the front end of the control unit) will cause the upper surface 104 to rotate in the +a direction, and sliding the actuator in the +z direction (towards the rear end 202a of the control unit) will cause the upper surface to rotate in the −a direction. In other words, when the control unit is in rotation mode, and is oriented substantially parallel to the z axis, sliding the actuator 206 towards the front end 202b of the control unit causes the upper surface 104 to rotate clockwise when viewed from the side (as in FIG. 6A).

In an alternative embodiment, as shown in FIG. 6C, the actuator 206 is rotatable with respect to the control unit 200 and/or to the track 208. If the control unit is oriented substantially parallel with respect to the z axis, then rotating the actuator 206 in the +a direction will cause the upper surface 104 of the upper surface 104 of the patient support to rotate in the +a direction. Similarly, rotating the actuator in the −a direction will cause the upper surface 104 of the patient support to rotate in the −a direction.

FIGS. 7A and 7B show how the upper surface 104 of the patient support 102 may be rotated in the +b and −b directions, about the y axis. In FIG. 7A, the upper surface 104, shown in an end view, is oriented with its longitudinal axis parallel to the y axis. With rotation mode selected, the actuator 206 can be used to rotate the upper surface 104 in the +b and −b directions. In this embodiment, with the control unit 200 oriented as shown in FIG. 7B, along the x axis, and with the front end 202b aimed in the +x direction, then sliding the actuator in the +x direction (towards the front end of the control unit) causes the upper surface to rotate in the +b direction. Conversely, sliding the actuator 206 along the track 208 in the −x direction (towards the rear end 202a of the control unit 200, as shown in FIG. 7B) causes the upper surface 104 to rotate in the −b direction, towards the position shown by the dashed line in FIG. 7A.

In this embodiment, orienting the control unit 200 in the opposite way, by aiming the front end 202b in the −x direction, does not change the functionality of the actuator 206; with the control unit 200 oriented in this way (that is with the front end 202b aimed in the −x direction), sliding the actuator in the −x direction (towards the front end of the control unit) will cause the upper surface 104 to rotate in the +b direction, and sliding the actuator in the +x direction (towards the rear end 202a of the control unit) will cause the upper surface to rotate in the −b direction. In other words, when the control unit is in rotation mode, and is oriented substantially parallel to the x axis, sliding the actuator 206 towards the front end 202b of the control unit causes the upper surface 104 to rotate clockwise when viewed from the end (as in FIG. 7A).

While, in the embodiments described above, orienting the control unit 200 in a particular way with respect to the patient support 102 causes the upper surface 104 to move in a particular direction, those directions, in other embodiments, may be reversed or varied. For example, orienting the control unit 200 such that it is parallel to the y axis may cause movement of the upper surface 104 along the x or z axes.

In an alternative embodiment, as shown in FIG. 7C, the actuator 206 is rotatable with respect to the control unit 200 and/or to the track 208. If the control unit is oriented substantially parallel with respect to the x axis, then rotating the actuator 206 in the +b direction will cause the upper surface 104 of the upper surface 104 of the patient support to rotate in the +b direction. Similarly, rotating the actuator in the −b direction will cause the upper surface 104 of the patient support to rotate in the −b direction.

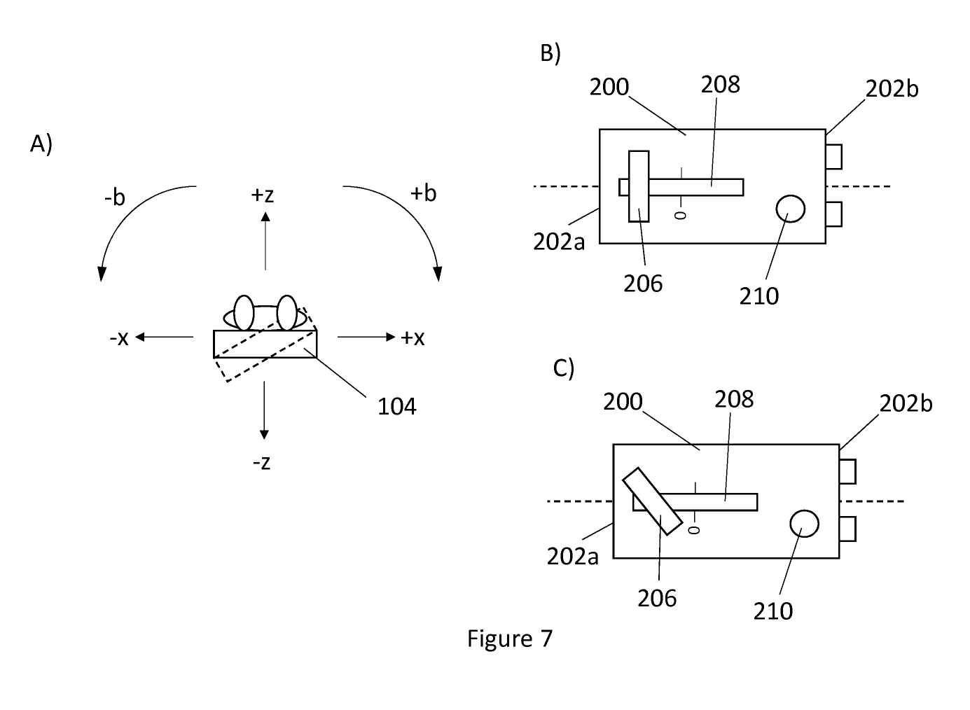

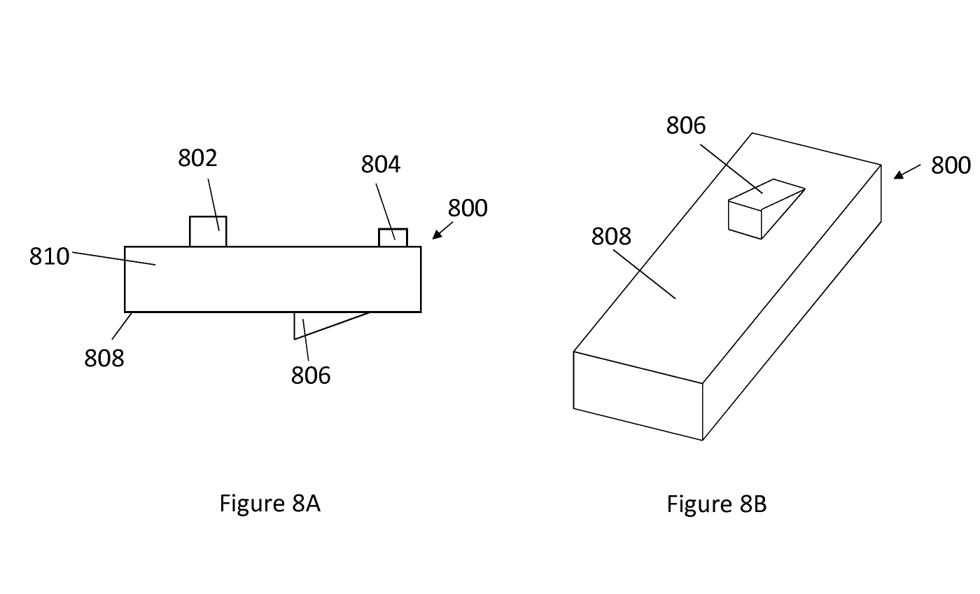

FIGS. 8A and 8B show a control unit 800 constructed in accordance with an alternative embodiment of the invention. FIG. 8A is a side view of the control unit 800 which includes an actuator 802 and a button 804, which correspond to the actuator 206 and the button 210 of the control unit 200 of FIGS. 2A and 2B. In this embodiment, however, the control unit 800 includes a spring-loaded switch 806 extending from a bottom wall 808 of a housing 810 of the control unit. The switch 806 functions as a “dead man's switch” so that functionality of the actuator 802 and the button 804 is only enabled if the switch 806 is pressed. The location of the switch 806 enables a user to press the switch with his or her finger while holding the control unit 800. Thus, a user holding the control unit 800 is able to press the switch 806 with his or her finger and, simultaneously, control the actuator 802 and the button 804 with his or her thumb. The switch 806 is spring-loaded such that, if the user removes his or her finger from the switch, for example if the user drops the control unit 800, and pressure on the switch is removed, then the switch rapidly opens, thereby preventing further movement of the upper surface 104 of the patient support 102.

So far, the invention has been described in terms of individual embodiments. However, those skilled in the art will appreciate that various embodiments of the invention, or features from one or more embodiments, may be combined as required. It will be appreciated that various modifications may be made to these embodiments without departing from the scope of the invention, which is defined by the appended claims.

1. A patient support system, comprising:
a patient support having a base and an upper surface, the upper surface moveable relative to the base in a direction along at least one of a first axis, a second axis orthogonal to the first axis, and a third axis orthogonal to the first and second axes; and
a control unit for controlling movement of the upper surface, the control unit defining a longitudinal axis;
wherein the direction of movement of the upper surface relative to the base is determined by the orientation of the longitudinal axis of the control unit relative to the patient support.

2. The patient support system according to claim 1, wherein, when the control unit is oriented such that its longitudinal axis is parallel to or within a defined angular range of the first axis, the upper surface is moveable along the first axis, when the control unit is oriented such that its longitudinal axis is parallel to or within a defined angular range of the second axis, the upper surface is moveable along the second axis, and when the control unit is oriented such that its longitudinal axis is parallel to or within a defined angular range of the third axis, the upper surface is moveable along the third axis.

3. The patient support system according to claim 2, wherein, when the control unit is oriented such that its longitudinal axis is outside any of the defined angular ranges, control of the movement of the upper surface by the control unit is prohibited.

4. The patient support system according to claim 2, wherein the defined angular range about each axis is a range of 30 degrees.

5. The patient support system according to claim 1, wherein the control unit comprises a first user interface for receiving a user input, and wherein the upper surface is configured to move in response to a user input received via the first user interface.

6. The patient support system according to claim 5, wherein the first user interface comprises a sliding input device, the sliding input device moveable along a track.

7. The patient support system according to claim 6, wherein the control unit has a front end and a rear end, and the sliding input device is movable between a front end of the track and a rear end of the track, the front end and the rear end of the track corresponding to the front end and the rear end of the control unit respectively.

8. The patient support system according to claim 7, wherein the sliding input device is biased towards a central position along the track.

9. The patient support system according to claim 8, wherein, when the control unit is oriented such that its longitudinal axis is parallel to or within the defined angular range of a particular one of the first, second and third axes, the direction of movement of the upper surface along the particular axis corresponds to a direction of movement of the sliding input device along the track.

10. The patient support system according to claims 6, wherein the sliding input device is rotatable with respect to the control unit.

11. The patient support system according to claim 10, wherein the sliding input device is configured such that rotating the sliding input device effects rotation of the upper surface relative to the base of the patient support.

12. The patient support system according to claim 1, wherein the upper surface is rotationally moveable relative to the base about at least one of the first axis, the second axis, and the third axis.

13. The patient support system according to claim 12, wherein the control unit further comprises a toggle switch, moveable between a first position and a second position, and wherein, when the toggle switch is in the first position, the first user interface is configured to effect linear movement of the upper surface and, when the toggle switch is in the second position, the first user interface is configured to effect rotational movement of the upper surface.

14. The patient support system according to claim 12, wherein, when the control unit is oriented such that its longitudinal axis is parallel to or within the defined angular range of the first axis, the upper surface is rotatable about one of the first, second or third axes, when the control unit is oriented such that its longitudinal axis is parallel to or within the defined angular range of the second axis, the upper surface is rotatable about another of the first, second or third axes, and when the control unit is oriented such that its longitudinal axis is parallel to or within the defined angular range of the third axis, the upper surface is rotatable about the other of the first, second or third axes.

15. The patient support system according to claim 1, wherein the control unit includes at least one transmitter and the patient support includes at least one receiver, and wherein the at least one transmitter is configured to communicate with the at least one receiver in order for the orientation of the control unit relative to the patient support to be determined.

16. The patient support system according to claim 15, wherein the at least one transmitter comprises an ultrasound transmitter and the at least one receiver comprises an ultrasound receiver.

17. The patient support system according to claim 15, wherein the at least one transmitter comprises an infrared transmitter and the at least one receiver comprises an infrared receiver.

18. The patient support system according to claim 15, wherein the at least one transmitter comprises an RFID transmitter and the at least one receiver comprises an RFID receiver.

19. The patient support system according to claim 1, wherein the control unit comprises an inertial measurement device for determining the orientation of the control unit relative to the patient support.

20. The patient support system according to claim 1, wherein the control unit comprises an activation switch moveable between a first position and a second position, and biased towards the second position, and wherein, when the activation switch is in the first position, functionality of the first user interface is enabled and, when the activation switch is in the second position, functionality of the first user interface is prevented.
